(19) United States
(12) Patent Application Publication
(10) Pub. No.: US 2021/0220260 A1
(43) Pub. Date: Jul. 22, 2021

(54) TOPICAL INJECTABLE COMPOSITION

(71) Applicant: JETEMA CO., LTD., Gangwon-do (KR)

(72) Inventors: Jae Young KIM, Seoul (KR); Jeong Sun NAM, Seoul (KR)

(73) Assignee: JETEMA CO., LTD.

(21) Appl. No.: 17/225,309

(22) Filed: Apr. 8, 2021

Related U.S. Application Data
(63) Continuation-in-part of application No. PCT/KR2020/001686, filed on Feb. 6, 2020.

(30) Foreign Application Priority Data
Feb. 8, 2019 (KR) 10-2019-0015232

(51) Int. Cl. A61K 9/00; A61K 31/575; A61K 9/16

(52) U.S. Cl. CPC A61K 9/0019; A61K 9/1611; A61K 9/1623; A61K 31/575

(57) ABSTRACT

The present invention relates to a dry powder formulation for injection of a DCA ingredient. The present invention is a dry powder formulation, which has excellent storage stability, has a pH of 8.2 or less so that, when dissolved in injection water, pain is lessened during injection by forming a pH environment closer to that of the human body than conventional injections, and allows DCA to dissolve well in injection water without precipitating.

### CROSS-REFERENCE TO RELATED APPLICATIONS

This application is a Continuation-In-Part application of PCT Application No. PCT/KR2020/001686, filed on Feb. 6, 2020, which claims priority to Korean Patent Application No. 10-2019-0015232, filed on Feb. 8, 2019. The entire disclosure of the applications identified in this paragraph is incorporated herein by references.

### TECHNICAL FIELD

The present invention relates to an injectable composition that includes deoxycholic acid. Specifically, it relates to a composition for dry powder formulation used in injections excellent for non-surgically removing fat from a person with locally deposited fat while minimizing pain, swelling or other side effects.

### BACKGROUND ART

[Fat Removal Procedure]

Locally deposited fat on the body like the face is not good from a cosmetic point of view. For example, with a double chin or deep cheeks, the face may look bigger.

Such localized fat deposits occur according to aging, lifestyle, or genetic factors. To overcome this, there are various exercise regimens and diets but with limited effectiveness. Thus, it has led to the advent of surgical therapy for local fat reduction.

The widely used surgical therapies include liposuction, lipoplasty, liposuction incision, and so forth. However, the surgical therapies have a limitation that they take several weeks to heal, a longer healing period for some patients, such as diabetics, and carry the risk of side effects, including excessive bleeding, internal organ damage, bacterial infection, scarring, or pain.

[PPC Injection for Fat Reduction]

Therefore, injections for local fat reduction have emerged as an alternative to surgical therapy. They consist of ingredients that decompose fat cells, and are used by injecting drugs into the subcutaneous fat layer. Lipostavil, a mixture of PPC and DCA, is a representative example of local fat reducing injection.

PPC is an essential phospholipid, a major component of cell membranes. In 1988, Dr. Sergio Magiori from Italy reported the PPC usage for the treatment of blepharoglobinoma, a disease involved in yellow fat deposits on the eyelids. In the mid-1990's, Dr. Patricia Rittes published the use of PPC for removing infraorbital fat tissue. This initiated widespread public interest in PPC usage for lipolysis treatment.

On the other hand, when used as an injection, the ingredients need to be dispersed in a small particle size. If ingredients prone to precipitation rather than solubilization are injected, large particles possibly block blood vessels, adversely affecting the blood flow of tissues around the blocked blood vessels or damaging or irritating the tissue to cause itching and pain. Yet, PPC is a waxy solid substance that is insoluble in water for injection.

For this reason, DCA is used as a solubilizing agent to disperse PPC to a particle size of 10 nm or less. DCA is one of the bile acids.

[DCA Injection for Fat Reduction]

In the off-label treatment of Lipostavil, a mixture of PPC and DCA, for local fact reduction, there are side effects, such as pain, local edema, erythema, hardening, paresthesia, itchiness, and burning sensation.

Korean Patent Publication No. 1217497 discloses a DCA sodium salt injection with the PPC content reduced or eliminated, claiming that PPC causes the above side effects and further that local fats can be emulsified solely with bile salts.

In April 2015, Kisera was approved as an injection for double chin reduction by the U.S. Food and Drug Administration. Approved as a specialized medication to improve the appearance and profile with a nonsurgical injection technique, Kybella is greatly expected in the facial plastic industry, following Botox, Natrelle, and Juvederm.

### PRIOR TECHNICAL DOCUMENTATION

### Patent Documents

Patent Document 1: Korean Registered Patent Publication No. 1217497
Patent Document 2: Korean Registered Patent Publication No. 1751585

### SUMMARY

### Technical Problem

Kybella, a DCA-based fat-removing injection, is in an alkaline environment with a pH value over 8.2. Korean Registered Patent Publication No. 1751585 attributes the alkaline environment of Kybella to the precipitation of DCA. In other words, according to the cited patent, DCA forms a precipitate in no more than four weeks in the pH range from 7.56 to 8.09, so it is unavailable as an injection for long-term distribution and stable only at pH 8.21-8.55, producing no precipitate.

It is a common sense that the pH and osmotic pressure of injections different from those of the human body cause pain. In other words, the pH of the human body is typically about 7.4-7.5, so pain is unavoidable when the pH of the injection is above 8.21. Despite this fact, however, the pH of Kybella is inevitably adjusted to the range causing pain due to the precipitation of DCA.

The inventors of the present invention have attempted to develop a composition for DCA injection having a pH value approximating to the pH of the human body in order to reduce injection pain. It is therefore an object of the present invention to develop an injectable composition that contains DCA sufficiently solubilized without precipitation when injected, has a pH value approximating to the pH of the human body, causing less pain, and exhibits availability for long-term distribution.

### Technical Solution

The present invention can solve the above-described problems by the following means. A feature of the present invention is that DCA or its salt is implemented into a dry powder formulation, which is a novel formulation that has never been marketed.

(1) A dry powder formulation for injection containing DCA or its salt.

(2) The dry powder formulation for injection according to (1), wherein the pH is 8.2 or less.

(3) The dry powder formulation for injection according to (1) or (2), wherein the pH is 8.0 or less.

(4) The dry powder formulation for injection according to any one of (1), (2) and (3), wherein the pH is 7.4-7.9.

(5) The dry powder formulation for injection according to any one of (1) to (4), wherein the dry powder formulation includes sugar or sugar alcohol as an excipient.

(6) The dry powder formulation for injection according to (5), wherein the sugar or sugar alcohol is mannitol.

(7) The dry powder formulation for injection according to any one of (1) to (6), wherein the dry powder formulation for injection is not required to contain a preservative.

(8) A use of the dry powder formulation for injection according to any one of (1) to (7) in the manufacture of a formulation for induction of lipolysis or treatment of an obesity disease.

(9) A use of the dry powder formulation for injection according to any one of (1) to (7) for induction of lipolysis or treatment of an obesity disease.

(10) A method for inducing lipolysis or preventing or treating an obesity disease that includes administering a therapeutically effective amount of the dry powder formulation for injection according to any one of (1) to (7) to a mammal, including a human being.

In the method according to any one of (8), (9) and (10), the obesity may include, but not limited to, local obesity.

In the method according to (8), the dry powder formulation for injection according to the present invention for the manufacture of a formulation for treating an obesity disease may include a pharmaceutically acceptable carrier or the like, and may further include other agents.

In the method according to (10), the term “therapeutically effective amount” refers to an amount of a drug effective for the treatment of an obesity disease, e.g., an amount of a dry powder formulation for injection administered to a biological individual subject, including any amount of the dry powder formulation for injection to make an effect of preventing occurrence or recurrence of an obesity disease, alleviating symptoms, inhibiting direct or indirect pathological consequences, preventing metastasis, lowering the rate of progression, alleviating or temporarily reducing the condition, or improving the prognosis. In other words, the therapeutically effective amount may be interpreted as encompassing all doses by which the dry powder formulation for injection can improve or cure the symptoms of the obesity disease.

The method for preventing or treating an obesity disease includes administering a dry powder formulation for injection not only to deal with the disease itself prior to the onset of the symptoms of the disease, but also to inhibit or avoid the symptoms of the disease. In the management of a disease, the prophylactic or therapeutic dose of a specific active ingredient varies depending on the nature and severity of the disease or condition and the route of administration of the active ingredient. The dose and frequency also vary depending on the age, weight and response of the individual patient. A suitable dosage regimen can be readily selected by those skilled in the art taking these factors into account. In addition, the treatment method of the present invention may further include administering a therapeutically effective amount of an additional active agent used to the treatment of an obesity disease together with the dry powder formulation for injection. The additional active agent may exhibit a synergistic or additive effect with the dry powder formulation for injection.

The mammal including a human being may include mammals, such as humans, monkeys, cows, horses, dogs, cats, rabbits, and rats.

### Effects of Invention

The present invention is stable for at least 30 months from the date of manufacture and excellent in storage stability even for a longer period of time. As the present invention has a pH value of 8.2 or less when dissolved in water for injection, it causes less pain than the conventional formulations and is clearly dissolved in the water for injection without causing precipitation of DCA.

In addition, the present invention can be easily dissolved in water for injection at high dissolution rate without any special stirring mechanism, causing little inconvenience in use.

In addition, the present invention has a manufacturing advantage such as good formation of a cake when making a dry powder formulation.

In addition, even if not containing a preservative particularly harmful to the human body, the present invention has no problem in terms of acceptance criteria for biological contamination or the like.

### BRIEF DESCRIPTION OF THE DRAWINGS

FIG. 1 presents the dissolution of a DCA ingredient itself (left) and the DCA dry powder formulation (right) of the present invention in water for injection at pH 7.0, where the images from top to bottom show before dissolution, 10 seconds after dissolution, and 30 seconds after dissolution, respectively.

FIG. 2 presents the dissolution of a DCA ingredient itself (left) and the DCA dry powder formulation (right) of the present invention in water for injection at pH 7.8, where the images from top to bottom show before dissolution, 10 seconds after dissolution, and 30 seconds after dissolution, respectively.

FIG. 3 presents images showing the appearance of DCA dry powder formulations according to the embodiments of the present invention, i.e., Examples 1, 2 and 3 from left to right.

FIG. 4 presents the dissolution of the DCA dry powder formulation of the present invention in water for injection, where the images from top to bottom show before dissolution, 10 seconds after dissolution, 30 seconds after dissolution, and 8 hours after dissolution, respectively.

### DETAILED DESCRIPTION

### Best Mode for Embodiment of Invention

In this specification, the term “DCA” refers to deoxycholic acid, which is sometimes interpreted as including all of deoxycholic acids or pharmaceutical salts thereof.

In this specification, the term “PPC” means phosphatidylcholine.

In this specification, the term “dry powder formulation” refers to a novel formulation of DCA devised by the inventors of the present invention as a solid formulation that is pulverized to a size suitable for reconstitution in water for injection.

The submental fat makes it difficult to have the face with a well-balanced and harmonious appearance, making the face look older and fatter than it actually is. It possibly appears irrespective of the body weight and has a great emotional impact in the negative way as it has established itself as one of the factors that bring about an inferiority complex connected to physical appearance. Accordingly, many consumers have requested improvement of double chins, but there is no appropriate non-surgical treatment alternative. In April of 2015, KYTHERA Biopharmaceuticals Inc. was approved by the U.S. Food and Drug Administration (FDA) for the sale of Kybella (Codename ATX-101), an injection for treatment of people with double chins. Following Botox, Natrel, and Jubiderm, Kybella is a specialty medicine that is capable of improving beauty only by injection without surgical operation and highly expected in the fields of facial plastic surgery. According to a phase-3 clinical trial carried out in North America, 68.2% of the ATX-101 test group agreed that their appearance improved, and 20.5% responded that their psychological impact was reduced. It was thus found that the administration of ATX-101 improved the feeling of happiness and self-esteem of the clinical trial subjects and greatly relieved the feeling of looking old or fat.

Kybella contains deoxycholic acid (DCA) as an active ingredient. DCA is a form of bile that destroys fat cells in the digestive tract. In fact, DCA has been an additive used as a solubilizing agent for insoluble phosphatidylcholine (PPC) in PPC injections, where the PPC is a lipid component for dissolving fat. In the 2000s, an off-label injection of PPC was used to reduce the subcutaneous fat accumulated in the abdominal area, under the chin, thighs and arms. It was later revealed that what physically destroys the cell membrane of adipocytes (fat cells) and induces adipocytolysis was not PPC but DCA, which was used as a solubilizing agent for PPC injection. KYTHERA used DCA as a main ingredient to develop a cell lysis agent for improving the appearance of people with a moderate to severe buildup of fat forming a convex or chubby profile under the chin.

Yet, Kybella can cause pain at the injection site as a side effect. The reason is that the pH of Kybella is too alkaline.

KYTHERA recognized that DCA with a concentration of 1% w/v was stable without precipitation at pH 8.2-8.5 but precipitated and became insoluble at a pH value approximating to the pH of the human body. It is necessary for the injectable formulation to have all the ingredients evenly dispersed and solubilized, because precipitation of any ingredient may block the blood vessels to cause problems. Hence, KYTHERA inevitably made an alkaline environment with pH about 8.2 or above in order to inhibit the precipitation of DCA. As a result, Kybella is a preparation that inevitably causes pain where the injection was given.

The inventors of the present invention have contemplated a way to reduce the injection pain, that is, to provide the environment with the lower pH value and surprisingly found it out through experiments that DCA constituted as a dry powder formulation can be injected without forming a precipitate even at a pH value approximating to the pH of the human body, thereby completing the present invention.

The present invention is characterized in that it is a dry powder formulation, which is a novel DCA-associated formulation that is hitherto unmarketed. The dry powder formulation for injection according to the present invention is prepared in the form of a solid pulverized into a size that can be generally referred to as a size of powder during distribution, and dissolved in water for injection when in use. The particle size of the present invention is not limited to a specific size and may be any size suitable for rapidly dissolving in water for injection.

The dry powder formulation for injection according to the present invention has a pH value of 8.2 or less, preferably 8.0 or less, and more preferably 7.4 to 7.9. When dissolved in general neutral water for injection, the dry powder formulation for injection according to the present invention similarly has a pH value of 8.2 or less, preferably 8.0 or less, and more preferably 7.4 to 7.9. As the novel dry powder formulation offers a pH environment more approximating to the pH of the human body than the conventional products, it causes far less pain when injected.

The inventors of the present invention also confirmed the fact that DCA forms a precipitate at pH values of 8.2 or less. If a raw material itself has a property of forming a precipitate, changing the formulation alone does not make any change in that property and the material will still form a precipitate. To the knowledge of the inventors of the present invention, there has never been an attempt to change the formulation of DCA in the prior art. Particularly, a DCA formulation prepared in the form of dry powder adds the hassle of dissolving in water for injection when used as an injectable formulation. It is therefore not general to change the formulation of DCA into a solid form.

Although the exact principle is unknown, DCA itself forms an unstable precipitate at pH 8.2 or less, but the DCA formulation in the dry powder form does not precipitate during reconstitution in water for injection. A detailed description will be given in the following example.

The dry powder formulation of the present invention comprises a main component, an excipient, and a pH adjuster. Of course, other additives may also be included within a range that does not impair the effects of the present invention.

In the present invention, the main component may be DCA or its salt without limitation. For example, DCA is used in an embodiment of the present invention.

From the viewpoint of reconstitution, the main component is preferably contained in an amount of 5 to 50% with respect to the total dry weight of the dry powder formulation. More preferably, the content of the main component is 10 to 20% with respect to the total dry weight of the dry powder formulation.

In the present invention, the excipient, if not specifically limited, may be a sugar or a sugar alcohol in consideration of the properties that it can control isotonicity, guarantee long-term storage stability and rapidly dissolve in water for injection during reconstitution. Examples of the sugar or the sugar alcohol may include glucose, fructose, sucrose, lactose, trehalose, maltose, sorbitol, mannitol, xylitol, or erythritol.

The excipient is preferably contained in an amount of 40 to 90% with respect to the total dry weight of the dry powder formulation in order to form an adequate dry powder cake. More preferably, the content of the excipient is 70 to 80% with respect to the total dry weight of the dry powder formulation.

A step of reconstitution in water for injection is necessary in order to use the dry powder formulation. It can be a considerable inconvenience to put the dry powder formulation into practical use when the reconstitution step is complicated and clear dissolution is achieved only by agitation with a special agitation mechanism for a long time.

In this regard, the DCA dry powder formulation of the present invention can be easily dissolved in water for injection without a special agitation mechanism, causing no hassle in use. Particularly, using mannitol as an excipient can greatly improve the dissolution rate and the convenience of dissolution in water for injection.

Besides, the mannitol is more favorable in forming a cake in the preparation of the dry powder formulation.

In the present invention, the pH adjuster is not specifically limited as long as it is a component capable of adjusting the pH to 8.2 or less. Examples of the pH adjuster may include hydrochloric acid, citric acid, lactic acid, malic acid, acetic acid, phosphoric acid, tartaric acid, sodium carbonate, sodium bicarbondate, or sodium hydroxide. Preferably, the pH adjuster may be a combination of sodium hydroxide and hydrochloric acid.

The content of the pH adjuster depends on the desired pH range.

The present invention may include a general amount of other additives normally used in dry powder formulations. For example, the present invention does not preclude the inclusion of stabilizers (or surfactants), such as amino acids or chelating agents, or dissolution aids, such as propylene glycol, that are commonly used in the dry powder formulations for injection.

Even if not including a separate stabilizer or a dissolution aid, the dry powder formulation of the present invention allows DCA to dissolve clearly without forming a precipitate. In other words, the present invention enables a main component of DCA to dissolve clearly rather than turbidly in the preparation of dry powder formulations or the reconstitution of dry powder formulations in water for injection even when the dry powder formulations use neither a stabilizer nor a dissolution aid.

The present invention is adequate for approval criteria in terms of biological contamination even though it does not include a preservative that is commonly used in injections. For example, the present invention has an advantage of not having to contain preservatives, while the conventional products necessarily contain preservatives. For reference, it is well known that overdosing of preservatives is harmful to the human body, so it is desirable to reduce the use of preservatives as possible.

The dry powder formulation that is a novel DCA formulation of the present invention can be prepared as follows.

Firstly, an excipient is dissolved in water for injection, and DCA is added as a main component, forming a suspension. Then, a basic pH adjuster is slowly added until the solution appears clear. The pH adjuster can be added as much as to make the pH about 10 or above. When the DCA is completely dissolved, an acidic pH adjuster is slowly added to the solution of the completely dissolved DCA to adjust the pH of the DCA solution to 8.2 or below. The final volume is marked, and a dry powder processing is then performed. The dry powder processing is conducted with a general dry powder processing machine according to general dry powder processing methods to prepare a dry powder through drying and pulverization.

The dry powder formulation has to be dissolved in water for injection before used as an injection. The water for injection may be general neutral sterile water for injection. The typical pH of the water for injection commercially available may range from about 7.0 to about 7.8.

In this regard, the amount of the dry powder formulation may be the same as that of the conventional product, Kybella. For example, the active ingredient may be formulated at a concentration of 0.4% w/v to 2% w/v, which is reported to be effective in eliminating a buildup of fat in the body. Preferably, the active ingredient may be formulated at a concentration of 0.5% w/v to 1% w/v.

Hereinafter, the present invention will be described in further detail with reference to the following examples, which are not construed to limit the scope of the present invention.

### Examples

A dry powder formulation was manufactured according to the composition given in Table 1 below (Example 1).

TABLE 1

| Ingredients | Contents | |
|---|---|---|
| Deoxycholic acid | 50.0 | mg |
| D-mannitol | 273.7 | mg |

| | |
|---|---|
| Sodium hydroxide | q.s. |
| Hydrochloric acid | q.s. |

1. After dissolving D-mannitol in 70% of the final volume of the water for injection, add deoxycholic acid into a suspension.

2. Gradually add 10 M sodium hydroxide until the solution becomes clear (at least pH 10).

3. To the completely dissolved deoxycholic acid solution, gradually add 1 M hydrochloric acid to adjust the pH value to 7.8.

4. Mark the final volume (5 mL).

5. Dry the powder product, where Example 1 has a pH value of 7.8.

[Precipitation Test]

The DCA ingredient itself (Comparative Example) and the dry powder formulation (Example 1) as a novel DCA formulation manufactured according to the present invention were dissolved in water for injection at pH 7.0 and 7.8. The solution was agitated simply by hand without a separate device in the same way that a dry powder formulation is generally dissolved in water for injection prior to administration by injection in a clinic.

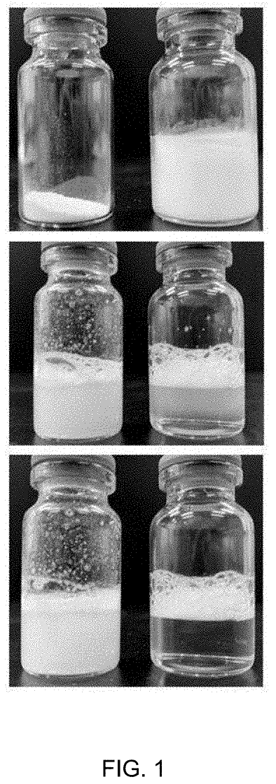

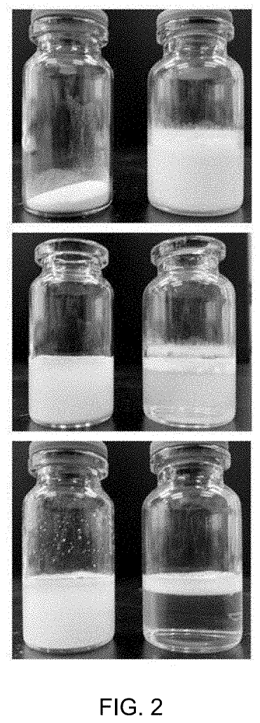

As a result, as shown in FIGS. 1 and 2, the DCA ingredient itself did not dissolve easily but formed a precipitate, while the DCA dry powder formulation dissolved sufficiently in 30 seconds and formed no precipitate. Despite the similar pH environment, the DCA ingredient itself and the dry powder formulation of the present invention were noticeably different in the degree of dissolution from each other.

FIG. 1 shows the dissolution in water for injection at pH 7.0, and FIG. 2 shows the dissolution in water for injection at pH 7.8. In FIGS. 1 and 2, the images from top to bottom show before dissolution, 10 seconds after dissolution, and 30 seconds after dissolution, respectively.

Particularly, the DCA dry powder formulation had a pH value of about 7.8, so even if it was dissolved to reconstitute in the water for injection at pH 7.0 and pH 7.8, the pH value was maintained at 8.2 or below. Unlike the conventional perception, the DCA dry powder formulation was rapidly dissolved without precipitation of DCA.

[Stability Test]

The dry powder formulation (Example 1) as a novel DCA formulation manufactured according to the present invention was evaluated in regards to the stability under the conditions as follows.

TABLE 2

| Items | Conditions |
|---|---|
| Storage container | Vial: Borosilicate glass |
| | Rubber stopper: Chlorobutyl, medical |
| Storage conditions | Long-term storage conditions: 25 ± 2° C., 60 ± 5% |
| | Accelerated conditions: 40 ± 2° C., 75 ± 5% |
| Storage period | 3 months |

As a result, the Example 1 according to the present invention as follows had neither generation of related substances nor pH variations even under the long-term storage conditions and accelerated conditions. In addition, it did not have any change in appearance occasionally occurring in some solid formulations.

TABLE 3

| | | After 3 months | After 3 |
|---|---|---|---|
| | | under long-term | months under |
| | | storage conditions | accelerated conditions |
| Items | Initial | (25 ± 2° C., 60 ± 5%) | (40 ± 2° C., 75 ± 5%) |
| Appearance | Suitable | Suitable | Suitable |
| Content | 96.7% | 96.7% | 97.3% |
| Related substance | N/D | N/D | N/D |
| pH | 7.8 | 7.8 | 7.8 |

[Dissolution Rate and Manufacturability]

A comparison was made in regards to the appearance properties after reconstitution and manufacture as a function of the type of the excipient.

A dry powder formulation was manufactured under the same conditions of Example 1, excepting that the excipient was replaced by lactose (Example 2) or fructose (Example 3).

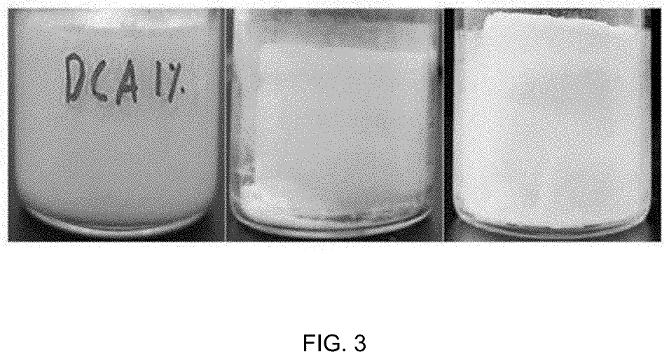

As a result, both the dry powder formulations were manufactured so that the appearance properties were more stable particularly when using mannitol in Example 1 (Refer to FIG. 3). In other words, as can be seen from FIG. 3 in which the images from left to right show the cases of using mannitol (Example 1), lactose (Example 2) and fructose (Example 3), respectively, all the examples had a cake formation without any issue, but the Example 1 using mannitol was the best in the degree of cake formation, suggesting that the use of mannitol resulted in the highest manufacturability.

The rate of dissolution upon reconstitution and the precipitation after dissolution were determined.

Water for injection was added to each of the Examples 1, 2 and 3, and after agitation for 5 seconds, the Examples 1, 2 and 3 were measured in regards to the degree of dissolution over time and stood at the room temperature for 8 hours to determine whether a precipitate was formed or not.

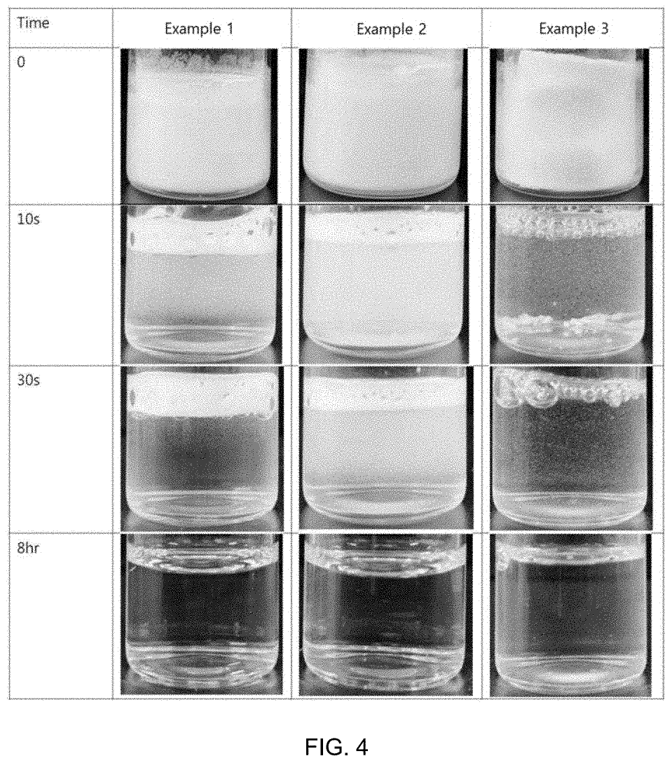

As a result, all of the Examples 1, 2 and 3 were completely dissolved in 30 seconds. Particularly, the Example 1 using mannitol was most quickly and completely dissolved. Further, none of the Examples 1, 2 and 3 formed a precipitate over 8 hours of dissolution (Refer to FIG. 4).

Industrial Availability

The present invention is directed to a dry powder formulation for injection that includes DCA or its salt. Specifically, it relates to a composition of a dry powder formulation used in injections excellent for non-surgically removing fat from a person with locally deposited fat while minimizing pain, swelling, or other side effects.

The dry powder formulation for injection containing DCA or its salt according to the present invention is stable for at least 30 months from the date of manufacture and excellent in storage stability even for a longer period of time. As the present invention has a pH value of 8.2 or less when dissolved in water for injection, it causes less pain than the conventional formulations and is clearly dissolved in the water for injection without causing precipitation of DCA.

What is claimed is:

1. A dry powder formulation for injection comprising DCA or a salt thereof.

2. The dry powder formulation for injection according to claim 1, wherein the dry powder formulation for injection has a pH value of 8.2 or less.

3. The dry powder formulation for injection according to claim 1, wherein the dry powder formulation for injection comprises sugar or sugar alcohol as an excipient.

4. The dry powder formulation for injection according to claim 1, wherein the dry powder formulation for injection is not required to comprise a preservative.

5. A method for inducing lipolysis or treating an obesity disease, the method comprising:
administering a therapeutically effective amount of the dry powder formulation for injection according to claim 1 to a mammal.

6. The method of claim 5, wherein the mammal is a human being.
